(19) United States
(12) Patent Application Publication
(10) Pub. No.: US 2026/0065622 A1
(43) Pub. Date: Mar. 5, 2026

(54) DIAGRAM ANALYSIS USING VISUAL LANGAUGE MODELS FOR MEDICAL DECISION MAKING

(71) Applicant: NEC Laboratories America, Inc., Princeton, NJ (US)

(72) Inventors: Yiyou Sun, Madison, WI (US); Wei Cheng, Princeton Junction, NJ (US); Haifeng Chen, West Windsor, NJ (US); Xujiang Zhao, Hillsborough, NJ (US)

(21) Appl. No.: 19/298,730

(22) Filed: Aug. 13, 2025

Related U.S. Application Data
(60) Provisional application No. 63/687,422, filed on Aug. 27, 2024.

(51) Int. Cl. G06V 10/26; G06T 7/11; G06T 7/187; G06T 7/50; G06V 10/46; G06V 10/82; G06V 20/62; G16H 50/20

(52) U.S. Cl. CPC G06V 10/267; G06T 7/11; G06T 7/187; G06T 7/50; G06V 10/46; G06V 10/82; G06V 20/62; G16H 50/20; G06T 2207/20021; G06T 2207/20084; G06T 2207/30004; G06V 2201/03

(57) ABSTRACT

Methods and systems for image analysis include initializing a set of initial regions that segment an input image. The initial regions are split into split regions. The split regions are merged into combined regions. Image analysis is performed on the combined regions using a visual language model, responsive to a query. An action is performed responsive to the image analysis in a downstream task.

### RELATED APPLICATION INFORMATION

This application claims priority to U.S. Patent Application No. 63/687,422, filed on Aug. 27, 2024, incorporated herein by reference in its entirety.

### BACKGROUND

### Technical Field

The present invention relates to visual language models and, more particularly, to diagram analysis.

### Description of the Related Art

Visual language models combine language understanding and image processing, with broad applicability in multimodal tasks such as navigation and answering questions. However, the perceptual capabilities of visual language models are still limited, as they often fail to discern fine visual details, are easily misled by visual distractors, struggle with understanding visual relationships, and may hallucinate non-existent objects.

### SUMMARY

A method for image analysis includes initializing a set of initial regions that segment an input image. The initial regions are split into split regions. The split regions are merged into combined regions. Image analysis is performed on the combined regions using a visual language model, responsive to a query. An action is performed responsive to the image analysis in a downstream task.

A system for image analysis includes a hardware processor and a memory that stores a computer program. When executed by the hardware processor, the computer program causes the hardware processor to initialize a set of initial regions that segment an input image, to split the initial regions into split regions, to merge the split regions into combined regions, to perform image analysis on the combined regions using a visual language model, responsive to a query, and to perform an action responsive to the image analysis in a downstream task.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram of an image analysis system with image segmentation, in accordance with an embodiment of the present invention;

FIG. 2 is a block/flow diagram of a method for image analysis that includes region splitting and region merging, in accordance with an embodiment of the present invention;

FIG. 3 is an exemplary diagram that has been split into regions, in accordance with an embodiment of the present invention;

FIG. 4 is a block diagram of a healthcare facility that uses image analysis with region splitting for medical decision making, in accordance with an embodiment of the present invention;

FIG. 5 is a block diagram of a computing device that can perform image segmentation and analysis, in accordance with an embodiment of the present invention;

FIG. 6 is a diagram of an exemplary neural network architecture that can be used to implement part of a visual language model, in accordance with an embodiment of the present invention; and

FIG. 7 is a diagram of an exemplary deep neural network architecture that can be used to implement part of a visual language model, in accordance with an embodiment of the present invention.

### DETAILED DESCRIPTION OF PREFERRED EMBODIMENTS

An image input can be decomposed into regions, with a visual language model (VLM) processing the image in an iterative fashion. Visual elements can be sequentially inferred, rather than generating a response in a single pass. The image may be segmented into regions of interest, tagging each object in the image with a number along with a segmentation mask indicator, prior to VLM analysis.

However, segmentation accuracy may be limited by generalization issues, as methods trained on natural images may fail with out-of-distribution inputs such as scientific information. Small or thin objects, such as the lines or dots of a chart, may furthermore be difficult to detect. Segmentation may further be limited by the difficulty of inferring hidden shape parameters, such as the rotational angle of a rectangle or the slope of a line.

Certain features common to displays of scientific visual information may be used to improve segmentation. Unlike natural images, such diagrams tend to have areas of homogeneous color and structured patterns for visual elements, as they are manmade or generated by software. These features can be used to enhance the identification of meaningful structures within diagrams.

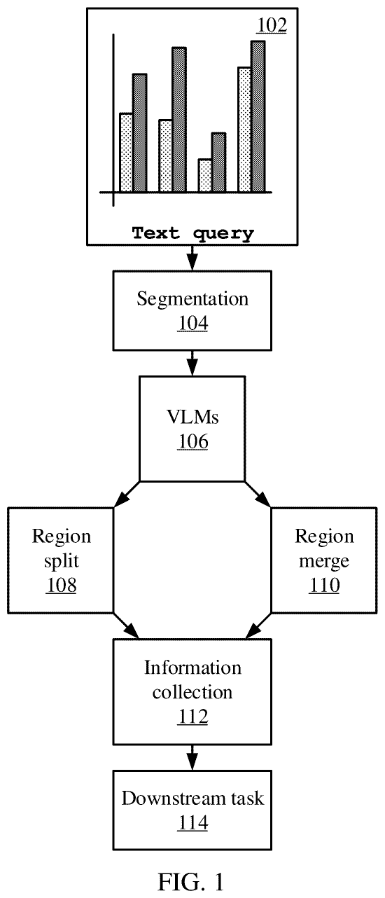

Referring now to FIG. 1, an exemplary VLM system is shown. An input 102 may include an image that shows a graph or another representation of medical or scientific information. A bar graph is shown, but it should be understood that the input 102 may include any appropriate representation of data. The input 102 may further include a text query, for example in the form of a natural language question about the contents of the image. In some cases the text query may ask for analysis of the information that is represented by the image.

Segmentation 104 divides the image of the input 102 into a set of regions, representing discrete visual elements or clusters of elements that are relevant to the text query. A VLM 106 performs information extraction for each of the regions. The extracted data is then aggregated and integrated into the VLM's prompt to form a comprehensive and contextually enriched input that improves the model's ability to generate accurate and relevant responses.

The segmentation 104 initializes the regions using connected components based on the observation that visual elements in scientific and medical diagrams exhibit homogeneous colors and structured pattens. This process begins with the conversion of the image Xᵢₘ into a binary image X_{bi}, which facilitates the separation of distinct visual components.

First the image may be converted into grayscale, and then into a binary format, using thresholding (e.g., Otsu's thresholding method), which automatically determines an optimal threshold value for separating the image into foreground and background. The resulting binary image X_{bi} includes clear distinctions between visual elements and the background.

Connected regions within X_{bi} are identified, for example using a method that assigns a unique label to each connected region based on 8-connectivity. The output, X_{fg}, represents the initial segmentation of the diagram where each unique label in the pixel location corresponds to a different visual component.

The inverse of the binary image (255-X_{bi}) may be processed to capture any visual components that may have been erroneously categorized in the original binary segmentation. This ensures that regions which are visually connected, but appear dark on a light background, are also identified as distinct entities. The resulting X_{bg} provides a complementary set of region masks.

The foreground X_{fg} and background X_{bg} are combined to create a unified region map X_{reg}. The term (X_{bg}>0)·offset ensures that the labels from X_{bg} are adjusted by a specified offset that guarantees there are no overlapping region labels, since both region maps begin the labeling from zero. Scientific and medical diagrams are thereby segmented into distinct and analyzable regions, setting the stage for further processing. However, this initial segmentation may not adequately separate connected objects that have overlapping elements, such as lines and bars.

Region splitting 108 may include a structured split and an unstructured split to address such elements. Before proceeding with specific region splitting, VLM 106 is used to preliminary identify potential structures or shapes within each main region, prioritizing regions based on area size. This may be performed with a prompt-based query to the VLM 106 which suggests possible structural categories. The VLM's response directly triggers the corresponding structure or shape detector. If the VLM 106 does not recognize the structure as one present in a current library, then an unstructured component split may be used. This discernment may be constrained to main regions with a large area size (e.g., area above a threshold value) and may be limited by a predefined number of attempts per image.

For regions identified as containing pre-defined structures, segmentation can be specialized to handle standard forms, such as geometric shapes and lines. A structure detector can be used to process each region independently based on a labeled mask Mᵢ. Structure detection analyzes Mᵢ and identifies sub-regions Mₛₕₐₚₑ that correspond to distinct structured elements within the region, along with parameters Sᵢ. The detected sub-regions are then split from the main region.

The pre-defined structures may include shapes that are frequently encountered in the analysis of scientific diagrams. Structure detection may extract accurate meta-information, such as the pixel location of rectangles, which may be used in subsequent diagram analysis.

After region splitting 108, there may be many identified regions and it may be impractical to extract information by querying the VLM 106 for each region individually. Region merging 110 may therefore be performed to reduce the number of regions. A structured merge may employ optical character recognition to detect and generate caption boxes within the image. Regions that are enclosed within text boxes may be regarded as related and may be merged into a single region. Thus for a text box that spans multiple regions, those regions may be merged together.

A set of heuristic rules may be used to merge regions based on visual patterns that follow fixed rules, such as dotted lines and background lines. Sequences of line segments may be considered part of a dotted or dashed line if the width and length of their boxes are approximately equal (within some threshold), the intervals between adjacent segment pairs are similar (within some threshold), and the angular difference between nearest pairs is less than a threshold (e.g., about 20 degrees). Background lines may be detected by identifying lines with horizontal or vertical orientations. For horizontal line segments, all y-axis positions are identified and the arithmetic mean is determined with e largest gap and the longest continuous segment. All such horizontal lines may be merged having y-axis values falling within the detected range. Vertical line segments may similarly be merged based on their x-axis positions.

Unstructured merging further consolidates the regions and may be particularly helpful when dealing with complex diagrams. Hierarchical clustering may be used to form region masks, with clustering being governed by a distance matrix D, each element Dᵢⱼ representing the distance between centroids of masks Mᵢ and Mⱼ. Hierarchical clustering may stop when the number of merged regions reaches a predefined budget B, which acts as a threshold to balance the number of queries made to the VLM 106 against the need to maintain a comprehensive understanding of the image.

Information collection 112 gathers meta-information from each region, including shape information Sᵢ and semantic information. The shape information is gathered from the structured split in region splitting 108 and includes details about the geometric properties of the region, such as contours, area, and perimeter. The semantic information may include the role and entity type of each region, along with a detailed description. The region may be visually highlighted with red in image Xᵢₘ and other regions may be faded to ensure that the VLM's attention is directed toward the relevant region during analysis. The highlighted image is sent to the VLM 106 using a prompt to extract detailed semantic information.

The collected shape and semantic information for each region is aggregated and appended to a master query X_{q}. This query, enriched with detailed regional metadata, is used to generate a response from the VLM 106. Each piece of collected information contributes to the formulation of the query and influences the VLM's output. Systematic collection of shape and semantic information helps the VLM recognize physical attributes of diagram components as well as their contextual and functional utility. Using this information, a downstream analysis task 114 can be performed.

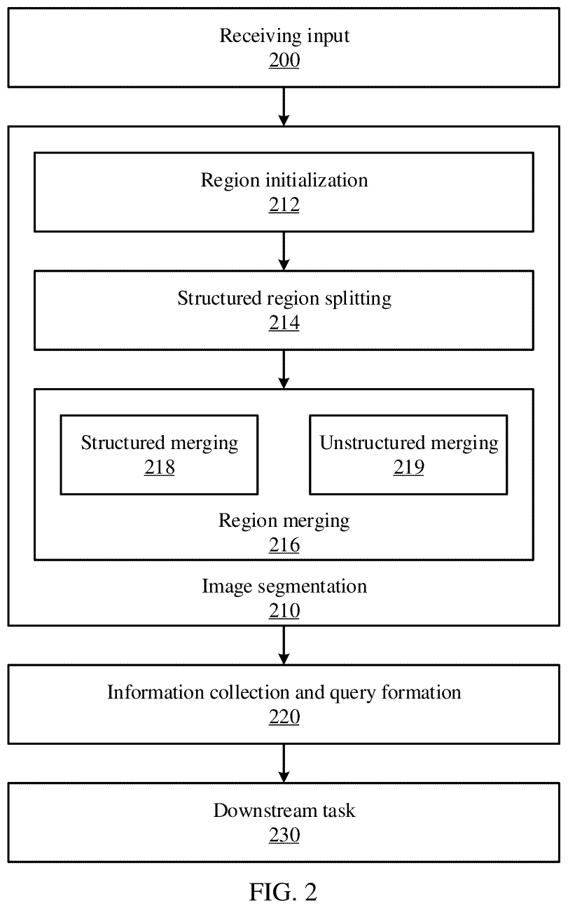

Referring now to FIG. 2, a method for performing a VLM task is shown. Block 200 receives an input that includes an image, such as a visual representation of scientific or medical data. Block 210 then segments the image into regions.

The segmentation includes a region initialization 212 that breaks the image into an initial set of regions, for example using thresholding to separate regions from one another. Block 214 then performs region splitting that includes structured splitting, capturing potential regions that the thresholding missed. The structured splitting may identify shapes within the image that match a predetermined set of structures, such as rectangles, ellipses, and lines. Sub-regions detected in this structured manner can be split from the region they were previously assigned to.

Region merging 216 then recombines the split regions into larger regions that can be processed together by the VLM. Region merging may include structured merging 218 and unstructured merging 219. The structured merging 218 detects and generates caption boxes within the image and further uses heuristic rules to merge regions based on visual patterns that follow fixed rules. The unstructured merge 219 may use hierarchical clustering on the region mask, based on a distance between centroids of the regions. Region merging 216 may continue until a number of regions reaches a predetermined threshold value.

The result of the splitting 214 and merging 216 is a set of regions that represent semantically significant structures within the image. Information collection and query formation 220 collects information from these regions, for example regarding the shapes shown in them and any semantic content encoded in them. Block 220 then collects the shape and semantic information for each region and creates a query for a VLM, for example appending it to the text query that was part of the input.

Block 230 performs a downstream task using the query. For example, the input image may include medical information relating to a patient. In one particular example, the input image may show the patient's health measurements taken over a period of time, such as heart rate, blood pressure, body temperature, cholesterol, etc. The query may ask the VLM to perform some analysis of this information, identifying trends or performing a statistical analysis of the data. This may be used when a graphical representation of the patient's past measurements is available, but the raw data is not. The downstream task 230 may thus include executing the query using a VLM and then performing diagnosis and treatment actions for the patient based on the analysis.

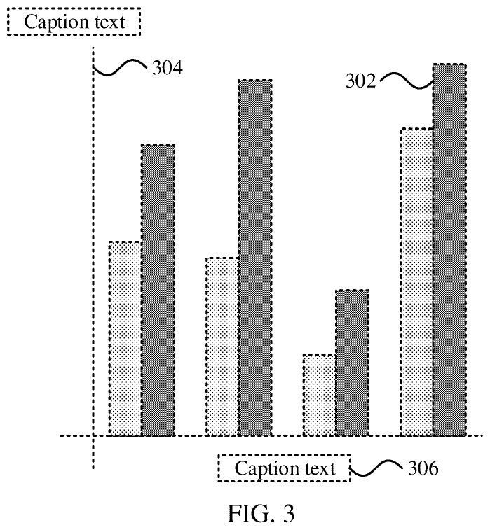

Referring now to FIG. 3, regions of an exemplary diagram are shown. The dotted lines indicate that geometric shapes 302 have been separated from one another, that the lines 304 have been identified, and that caption text 306 has been separated into respective regions for captions and other such markings.

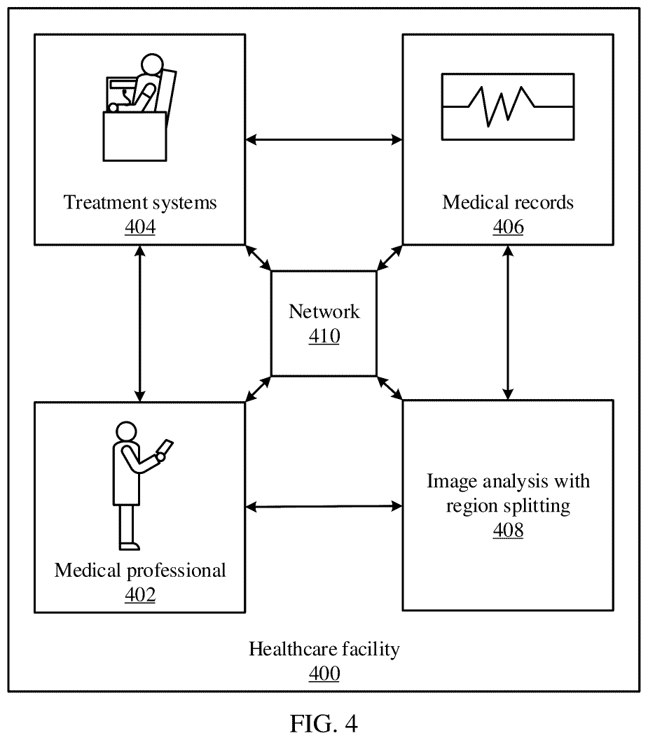

Referring now to FIG. 4, a diagram of image analysis with region splitting is shown in the context of a healthcare facility 400. Image analysis with region splitting 408 may be used to extract information from patient records that are stored in a graphical format, for example having been transferred from another healthcare facility, and that extracted information may be used to identify the patient's health condition and aid in medical decision making.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information drawn from the image analysis with region splitting 408, the medical professionals 402 may then make medical decisions about patient healthcare suited to the patient's needs. For example, the medical professionals 402 may make treatment decisions based on a diagnosis generated by the image analysis with region splitting 408 and may prescribe particular medications, surgeries, and/or therapies that are appropriate to the diagnosis disease.

The different elements of the healthcare facility 400 may communicate with one another via a network 410, for example using any appropriate wired or wireless communications protocol and medium. Thus image analysis with region splitting 408 receives data from treatment systems 404, medical professionals 402, and from medical records 406, and analyzes images in the medical records 406 to extract information that is relevant to the patient's condition. The image analysis with region splitting 408 may further coordinate with treatment systems 404 in some cases to automatically administer or alter a treatment. For example, if the image analysis indicates a particular condition, the system may automatically implement the treatment, such as by initiating or halting the administration of a medication.

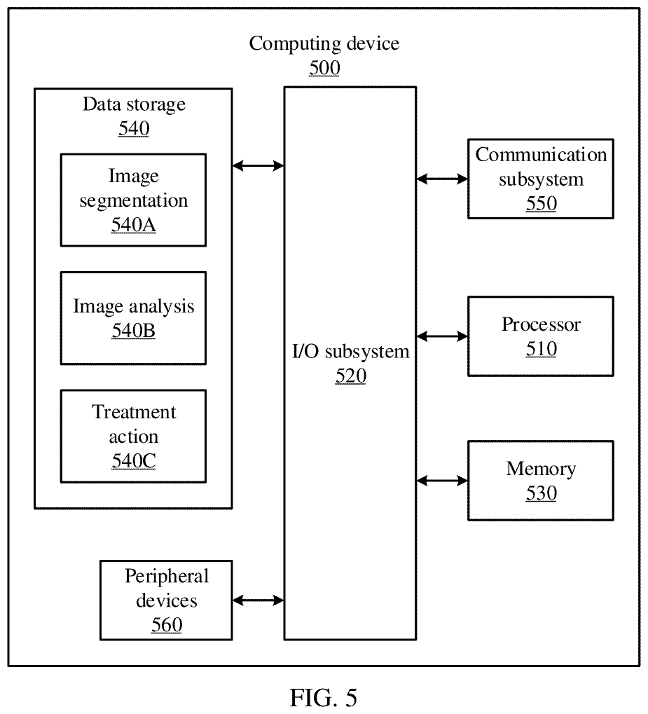

Referring now to FIG. 5, an exemplary computing device 500 is shown, in accordance with an embodiment of the present invention. The computing device 500 is configured to perform visual question answering.

The computing device 500 may be embodied as any type of computation or computer device capable of performing the functions described herein, including, without limitation, a computer, a server, a rack based server, a blade server, a workstation, a desktop computer, a laptop computer, a notebook computer, a tablet computer, a mobile computing device, a wearable computing device, a network appliance, a web appliance, a distributed computing system, a processor-based system, and/or a consumer electronic device. Additionally or alternatively, the computing device 500 may be embodied as one or more compute sleds, memory sleds, or other racks, sleds, computing chassis, or other components of a physically disaggregated computing device.

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for image segmentation, 540B for image analysis, and/or 540C for performing treatment actions. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

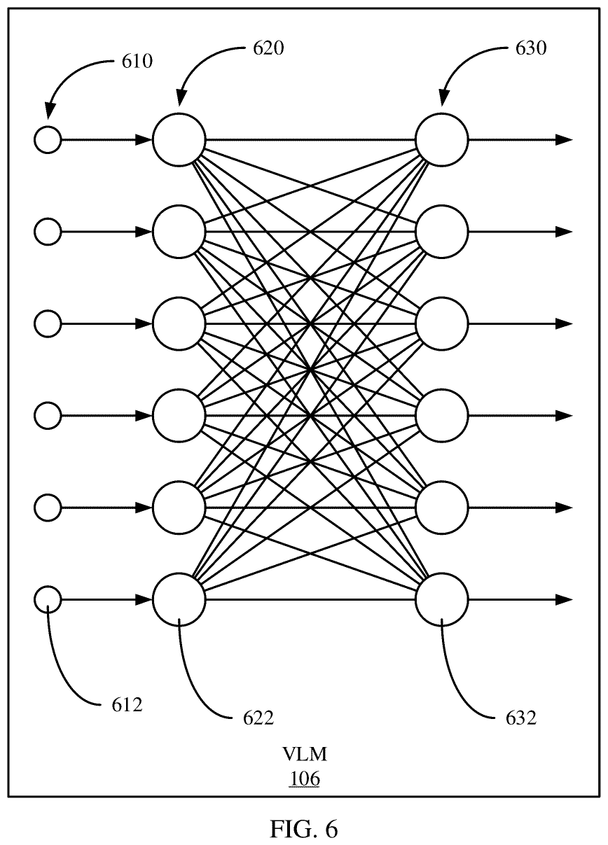

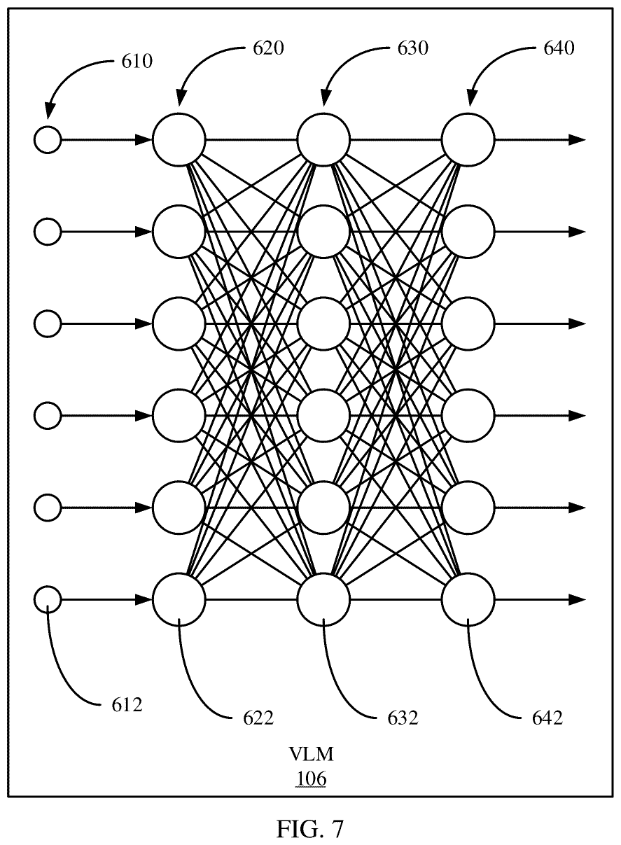

Referring now to FIGS. 6 and 7, exemplary neural network architectures are shown, which may be used to implement parts of the present machine learning models, such as the VLM 106. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer-readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for image analysis, comprising:
initializing a set of initial regions that segment an input image;
splitting the initial regions into split regions;
merging the split regions into combined regions;
performing image analysis on the combined regions using a visual language model, responsive to a query; and
performing an action responsive to the image analysis in a downstream task.

2. The method of claim 1, wherein splitting the initial regions into split regions includes structured splitting based on a set of predetermined shapes.

3. The method of claim 1, wherein merging the split regions into combined regions includes structured merging and unstructured merging.

4. The method of claim 3, wherein structured merging includes detecting a text box within the input image and merging regions that are spanned by the text box.

5. The method of claim 3, wherein structured merging includes heuristic rules that merge regions based on visual patterns, including dotted lines and background lines.

6. The method of claim 3, wherein unstructured merging includes hierarchical merging based on distances between centroids of the split regions.

7. The method of claim 1, further comprising extracting semantic information and shape information from the combined regions, wherein performing image analysis includes prompting the visual language model, which includes a machine learning model, with a prompt that includes an input query combined with the semantic information and the shape information.

8. The method of claim 1, wherein the input image shows medical data relating to a patient's health condition.

9. The method of claim 8, wherein the image analysis includes analysis of the medical data.

10. The method of claim 8, wherein the image analysis is used for medical decision making and wherein the action includes a treatment action that responds to a health condition of the patient.

11. A system for image analysis, comprising:
a hardware processor; and
a memory that stores a computer program which, when executed by the hardware processor, causes the hardware processor to:
initialize a set of initial regions that segment an input image;
split the initial regions into split regions;
merge the split regions into combined regions;
perform image analysis on the combined regions using a visual language model, responsive to a query; and
perform an action responsive to the image analysis in a downstream task.

12. The system of claim 11, wherein the split of the initial regions into split regions includes structured splitting based on a set of predetermined shapes.

13. The system of claim 11, wherein the merge of the split regions into combined regions includes structured merging and unstructured merging.

14. The system of claim 13, wherein structured merging includes detecting a text box within the input image and merging regions that are spanned by the text box.

15. The system of claim 13, wherein structured merging includes heuristic rules that merge regions based on visual patterns, including dotted lines and background lines.

16. The system of claim 13, wherein unstructured merging includes hierarchical merging based on distances between centroids of the split regions.

17. The system of claim 11, wherein the computer program further causes the hardware processor to extract semantic information and shape information from the combined regions, and wherein the image analysis includes prompting the visual language model, which includes a machine learning model, with a prompt that includes an input query combined with the semantic information and the shape information.

18. The system of claim 11, wherein the input image shows medical data relating to a patient's health condition.

19. The system of claim 18, wherein the image analysis includes analysis of the medical data.

20. The system of claim 18, wherein the image analysis is used for medical decision making and wherein the action includes a treatment action that responds to a health condition of the patient.
